(19) United States
(12) Patent Application Publication
(10) Pub. No.: US 2001/0023261 A1
(43) Pub. Date: Sep. 20, 2001

(54) Novel composition for the transdermal administration of drugs

(75) Inventors: Je-Phil Ryoo, Daejeon (KR); Mi-Suk Choi, Daejeon (KR); Jong-Kun Choi, Daejeon (KR)

Correspondence Address: David A. Einhorn, Esq., Anderson Kill & Olick, P.C., 1251 Avenue of the Americans, New York, NY, 10020, US

(73) Assignee: LG Chemical Limited.

(21) Appl. No.: 09/746,448

(22) Filed: Dec. 12, 2000

Related U.S. Application Data
(63) Continuation-in-part of application No. 09/293,255, filed on Apr. 16, 1999, now abandoned.
Application No. 09/293,255 is a continuation-in-part of application No. PCT/KR98/00013, filed on Jan. 23, 1998.

(30) Foreign Application Priority Data
Jan. 27, 1997 (KR) 1997-2233
Jun. 2, 1998 (KR) 1998-20378

(51) Int. Cl. A61K 47/10

(52) U.S. Cl. 514/772; 514/777

(57) ABSTRACT

A composition for the transdermal administration of a drug, comprising: 0.05 to 50 wt % of the drug based on the total weight of the composition; 1 to 30 wt % of an absorption promoter consisting of a diethylene glycol ether and a sorbitan ester in a weight ratio ranging from 1:4 to 4:1, based on the total weight of the composition; and a pharmaceutically acceptable adhesive matrix.

### CROSS REFERENCE TO RELATED APPLICATION

This application is a continuation-in-part (CIP) application of U.S. Ser. No. 09/293,255 filed on Apr. 16, 1999 which is a CIP application of PCT Patent Application No. PCT/KR 98/00013 filed on Jan. 23, 1998, designating the United States of America.

### FIELD OF THE INVENTION

The present invention relates to a composition for the transdermal administration of drugs, wherein a mixture of a diethylene glycol ether and a sorbitan ester is used as an absorption promoter; and to a transdermal formulation containing same.

### BACKGROUND OF THE INVENTION

The delivery of a drug through skin offers advantages over the conventional oral administration which is hampered by such problems as unpredictable rates of drug absorption, metabolic degradation of the drug and other adverse effects, e.g., gastrointestinal irritation caused by the drug itself or metabolites thereof. Namely, transdermal drug administration alleviates the aforementioned problems and delivers a drug at a controlled rate for an extended period of prescribed time due to increased bioavailability of the drug which is degraded in the digestive tract.

When a drug is transported through the skin, the horny layer of skin acts as a barrier against the permeation of the drug into the systemic circulation. Accordingly, various absorption promoters, or permeation enhancers, which facilitate the drug permeation through the horny layer, have been used in transdermal drug compositions. For example, U.S. Pat. Nos. 4,006,218; 3,551,554; 4,568,343; 4,746,515; 4,379,454; 4,906,463; 4,440,777; 4,783,450 and 5,212,199 disclose, as such absorption promoters, dimethyl sulfoxide (DMSO), dimethylformamide, polyethyleneglycol monolaurate, glycerol monolaurate, ethanol, propyleneglycol monolaurate, eucalyptol, lecithin and sorbitan esters.

Diethyleneglycol monoethyl ether, which has been used as a solubilizer in the formulation of naproxen, nitroglycerin, phenylbutazone and prazepam, was also reported to be effective as an absorption promoter in the transdermal administration of theophylline and prostaglandin E2 (Touitou, et al., International Journal of Pharmaceutics, 70, 159-166(1991); and Watkinson, A. et al., ibid, 74, 229-236(1991)).

Other absorption promoters disclosed in the prior art include: a mixture of linoleic acid and propyleneglycol (European Patent Publication No. 261429); and mixtures of N-(hydroxyethyl)pyrrolidone and methyl laurate, ethanol and glycerol monolaurate, diethylene glycol monoethyl ether and propyleneglycol monolaurate (U.S. Pat. Nos. 4,537,776; 4,764,379; and 4,973,468).

Further, as a formulation for the transdermal administration of a non-steroidal anti-inflammatory drug, U.S. Pat. No. 5,527,832 discloses a gel-type composition comprising Poloxamer® as a gelling agent; U.S. Pat. No. 5,505,956 describes a multi-layered patch formulation wherein the-water containing capacities of resins included in the layer increase from the skin-faced layer to the upper layer; and U.S. Pat. No. 5,656,286 teaches a simple matrix type patch using polyacrylate as an adhesive matrix.

The conventional transdermal formulations may be divided into three types: a reservoir type, a simple matrix type and a multi-layer lamination type. The simple matrix type formulation, as disclosed in U.S. Pat. Nos. 4,314,577; 4,438,139; and 4,839,174, comprises a drug dispersed in a layer made of a pressure-sensitive adhesive matrix. Such formulation can be produced at a low cost by a simple process. However, it has the problem that the rate of drug release is high in the initial stage and tapers off sharply thereafter.

Further, there exist the needs to deliver a large dose of a drug over an extended period using a simple matrix-type formulation. For this purpose, there have been the attempts to raise the drug content of the matrix to a level beyond the solubility of the drug in the matrix, i.e., supersaturate the matrix with the drug. However, supersaturation represents a thermodynamically unstable state and the drug in such formulation tends to form crystals. To alleviate this problem, crystal formation inhibitors, e.g., polyvinylpyrrolidone and polyvinylpyrrolidone-vinylacetate copolymer, have been used but the effectiveness thereof was observed to be marginal (Ma, X. et al., ibid, 142, 115-119 (1996)).

Accordingly, there exists a need for an improved matrix-type transdermal formulation of drugs having a steady and high rate of drug release over an extended period and also a high level of uncrystallized drug content. The present inventors have endeavored to develop an improved composition for the transdermal administration of drugs, the composition having a new absorption promoter which is capable of fulfilling the above needs. It has been unexpectedly found that a diethylene glycol monoalkyl ether and a sorbitan ester, each of which has been individually known as an absorption promoter having a limited effectiveness, provide a synergistic effect when combined, i.e., a mixture of a diethylene glycol ether and a sorbitan ester has been found to be a remarkably efficient absorption promoter for the transdermal transport of drugs.

### SUMMARY OF THE INVENTION

It is, therefore, an object of the present invention to provide an improved composition for the transdermal administration of a drug.

It is another object of the present invention to provide a transdermal formulation comprising said composition.

In accordance with one aspect of the present invention, there is provided a composition for the transdermal administration of a drug, comprising: 0.05 to 50 wt % of the drug based on the total weight of the composition; 1 to 30 wt % of an absorption promoter consisting of a diethylene glycol ether and a sorbitan ester in a weight ratio ranging from 1:4 to 4:1, based on the total weight of the composition; and a pharmaceutically acceptable adhesive matrix.

### BRIEF DESCRIPTION OF DRAWINGS

The above and other objects and features of the present invention will become apparent from the following description of the invention taken in conjunction with the following accompanying drawings, wherein:

FIG. 1 shows a schematic cross-sectional view of an embodiment of the inventive pharmaceutical formulation for the transdermal delivery of a drug;

FIG. 2 displays the time-dependent changes in the cumulative amount of estradiol transported across the skin of a hairless mouse as a function of the absorption promoter used;

FIG. 3 depicts the time-dependent changes in the cumulative amount of norethisterone acetate transported across the skin of a hairless mouse as a function of the absorption promoter used;

FIG. 4 illustrates the time-dependent changes in the cumulative amount of norethisterone acetate transported across human cadaver skin as a function of the absorption promoter used;

FIG. 5 represents the time-dependent changes in the cumulative amounts of ketoprofen transported across human cadaver skin as a function of absorption promoter used;

FIG. 6 exhibits the time-dependent changes in the cumulative amount of norethisterone acetate transported across human cadaver skin as a function of the contents of sorbitan monolaurate and diethylene glycol monoethyl ether; and

FIG. 7 compares the time-dependent changes in the cumulative amounts of ketoprofen transported across human cadaver skin in the experiments employing the inventive formulation and commercially available formulations.

### DETAILED DESCRIPTION OF THE INVENTION

The present invention provides a composition for the transdermal administration of a drug, comprising: the drug; a mixture of diethylene glycol ether and sorbitan ester, as an absorption promoter; and a pharmaceutically acceptable adhesive matrix.

Exemplary drugs for use in the composition of the present invention include estrogens, e.g., estradiol, ethynyl estradiol and estradiol ester; progestogens, e.g., norethisterone, norethisterone acetate, medroxyprogesterone acetate, desogestrel, gestaten and levonorgestrel; androgens, e.g., testosterone, testosterone propionate, testosterone enanthate, testosterone cypionate, methyltestosterone and dihydroepiandrosterone; non-steroidal anti-inflammatory drugs, e.g., ketoprofen, flurbiprofen, methylsalicylate, salicylic acid, ibuprofen, indomethacin, diclofenac sodium, flufenamic acid, naproxen, mefenamic acid, fenoprofen, fenclofenac and piroxicam; and a mixture thereof.

Total amount of the drug used in the inventive composition may range from 0.05 to 50 wt %, preferably, from 0.1 to 20 wt % based on the total weight of the composition. Especially, in case that an androgen is used as the drug, its amount ranges from 1 to 10 wt %, preferably, from 2 to 6 wt % based on the total weight of the composition; a non-steroidal anti-inflammatory drug, from 1 to 10 wt %, preferably, from 2 to 6 wt % based on the total weight of the composition; and a mixture of estrogen and progestogen, 0.05 to 5 wt % of estrogen and 1 to 10 wt % of progestogen, preferably, 0.2 to 2 wt % of estrogen and 1.5 to 6 wt % of progestogen, each based on the total weight of the composition.

The absorption promoter of the present invention is composed of a diethylene glycol monoalkyl ether and a sorbitan ester mixed in a weight ratio ranging from 1:4 to 4:1, preferably, from 1:2 to 2:1. Diethyleneglycol monoalkyl ethers which may be suitably used in the present invention include diethylene glycol monoethyl ether and diethylene glycol monomethyl ether, and suitable sorbitan esters include sorbitan monolaurate and sorbitan monooleate.

The total amount of the mixture of diethylene glycol ether and sorbitan ester used in the inventive composition may range from 1 to 30 wt %, preferably, from 5 to 25 wt % based on the total weight of the composition, wherein the weight ratio of said two components is kept within the aforementioned range.

The pharmaceutically acceptable adhesive matrix used in the present invention may be any of those known in the art, e.g., polyacrylate adhesives such as ethyl-, butyl- and 2-ethylhexyl acrylate, polyisobutylene and silicon rubber.

The composition of the present invention may further comprise flavoring agents, preservatives, anti-oxidants, stabilizers and pigments.

The transdermal formulation of a drug in accordance with another aspect of the present invention may be constructed using: a protective backing layer which is impermeable to the drug; a drug reservoir layer containing the aforementioned composition of the present invention, one side of which is laminated on the protective backing layer; and a peel layer attached to the other side of the drug reservoir layer, said peel layer being capable of protecting the composition from the environment until it is removed to bring the drug reservoir layer into contact with the skin. The formulation of the present invention may further comprise a supplementary adhesive layer which is selected from those known in the art, e.g., a ring-shaped adhesive layer sealably attached to the periphery of the drug reservoir layer and the protective backing layer.

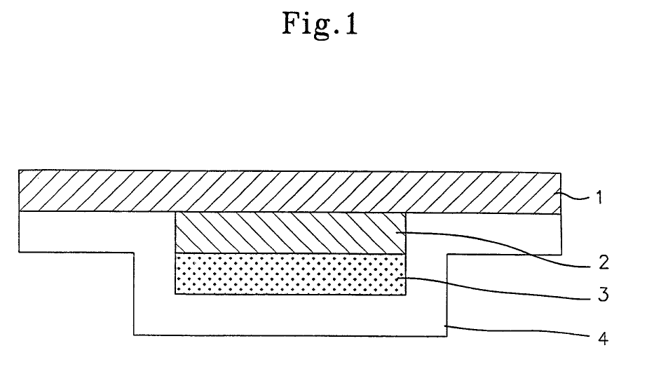

FIG. 1 shows a schematic cross-sectional view of an embodiment of the transdermal formulation of the present invention, which comprises peel layer (1), drug reservoir layer (2), protective backing layer (3) and supplementary adhesive layer (4).

The transdermal formulation of the present invention may be in the form of patches, plasters, pastes, cataplasma, gels, ointments and the like, which can be applied to the skin for a desired time period to achieve a desired blood level of drugs.

The inventive composition for the transdermal delivery of a drug has advantages in that: it is a simple matrix-type which can be prepared at a low cost; the use of the improved absorption promoter disclosed above makes it possible to maintain a high flux of the drug for an extended period, the apparent drug permeation rate following zero-order kinetics; and the formation of drug crystals is inhibited even at a high drug loading level owing to the use of said improved absorption promoter. The dosage administered to a patient by using the composition of the present invention can be controlled by adjusting the contents of the drug and the absorption promoter.

The following Examples are intended to further illustrate the present invention without limiting its scope.

Determination of Drug Permeation Rate through Skin

The flux, or the skin permeation rate, of a drug through a skin sample was determined by the following procedure.

A skin sample, either human cadaver skin or a skin piece excised from 6 week-old female hairless mouse, was installed in Valia-Chien diffusion cell (Crown Glass, U.S.A.) such that the stratum corneum of the skin faced outward from the cell, and then a transdermal formulation containing one or more drugs was fixed on the skin. 3.4 ml of physiological saline solution containing 40% of polyethyleneglycol 400 (Sigma Scientific Co.) was added to the cell and stirred for the whole period of experiment. Thereafter, 100 μl samples of the physiological saline solution were taken periodically and subjected to high performance liquid chromatography to determine the cumulative amount of the drug transported across the skin. The skin permeation rate (μg/cm²/hr) of the drug through the skin was calculated by regression analysis of the time-dependent cumulative amounts of the drug.

The process described above was repeated 3 times and averaged to define skin permeation rate of the drug.

### Reference Example 1

### Preparation and Testing of Transdermal Administration Compositions containing Conventional Absorption Promoters (Hairless Mouse Skin)

0.6 wt % of estradiol(ED), 3.0 wt % of norethisterone acetate (NETA) and an absorption promoter listed in Table 1 were added to a polyacrylate adhesive(Durotac 87-2074, National Starch Chem. Co.), and then the mixture was stirred sufficiently to dissolve the drugs in the adhesive.

The mixture thus obtained was poured onto an impermeable protective backing layer (Scotchpak 1109, 3M Co.) to coat a matrix layer having a thickness of 1000 μm. The resulting material consisting of the protective backing layer coated with the matrix layer was dried in an oven by raising the temperature stepwise from 60° C. to 120° C. The resulting material was cured in open air for 1 hour and a peel layer(Scotchpak 1012, 3M Co.) was laminated thereon. The resulting transdermal formulation was stored at room temperature.

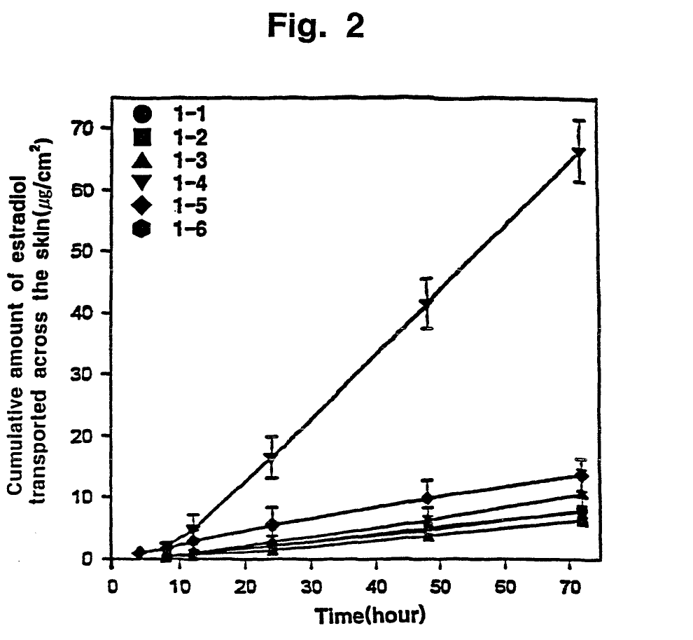

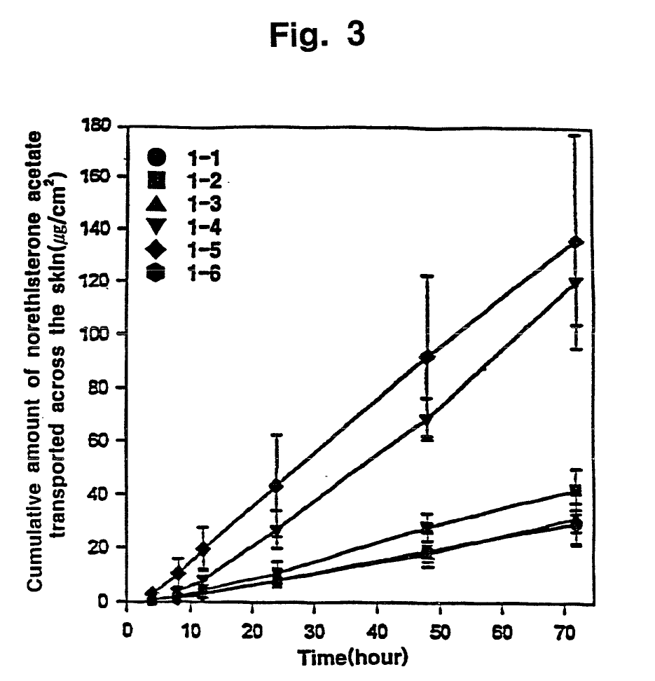

The permeation rates of the drugs across the skin of hairless mouse were determined and the results are shown in Table 1 and FIGS. 2 and 3.

TABLE 1
Permeation rates of ED and NETA across mouse skin in the
presence of conventional absorption promoter

| | | | Absorption | | |
|---|---|---|---|---|---|
| Reference | ED | NETA | Promoter | SPR* | SPR* |
| Example | (wt %) | (wt %) | (wt %) | (ED) | (NETA) |
| 1-1 | 0.6 | 3.0 | — | 0.12 | 0.43 |
| | | | | ±0.02 | ±0.09 |
| 1-2 | 0.6 | 3.0 | azone | 0.10 | 0.64 |
| | | | (10) | ±0.01 | ±0.08 |
| 1-3 | 0.6 | 3.0 | tricapryline | 0.16 | 0.49 |
| | | | (10) | ±0.05 | ±0.10 |
| 1-4 | 0.6 | 3.0 | sorbitan | 1.02 | 1.87 |
| | | | monolaurate | ±0.05 | ±0.17 |
| | | | (10) | | |
| 1-5 | 0.6 | 3.0 | squalene | 0.19 | 1.98 |
| | | | (10) | ±0.03 | ±0.48 |
| 1-6 | 0.6 | 3.0 | decanol | 0.11 | 0.45 |
| | | | (10) | ±0.01 | ±0.05 |

*SPR: Skin permeation rate across the skin
(mean ± SD(μg/cm²/hr))

FIGS. 2 and 3 show the time-dependent changes in the cumulative amounts of estradiol and norethisterone acetate transported across the skin as function of absorption promoter used.

As can be seen from Table 1 and FIGS. 2 and 3, the compositions containing sorbitan monolaurate(Reference Example 1-4) and squalene (Reference Example 1-5) as the absorption promoter exhibited high permeation rates. However, crystals of the drugs were observed in each of the above Reference Examples.

### Reference Example 2

### Preparation and Testing of Transdermal Administration Compositions Containing Conventional Absorption Promoters (Human Cadaver Skin)

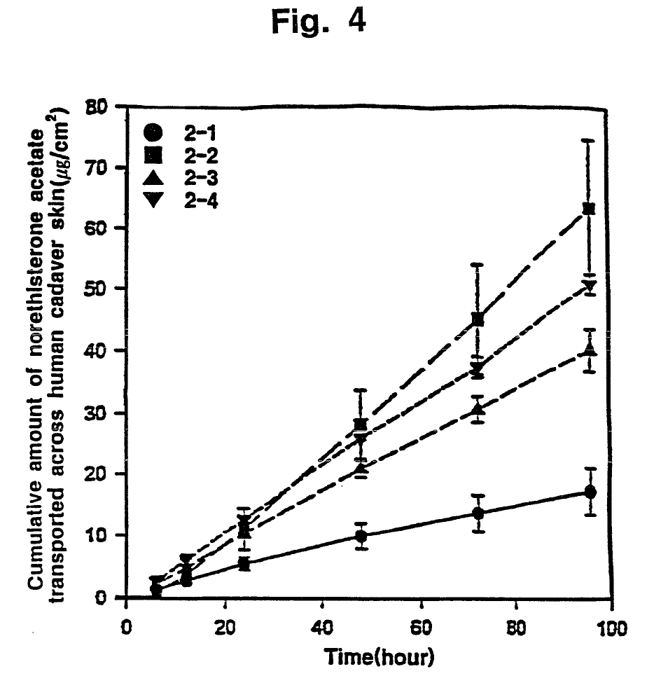

Four transdermal delivery compositions were prepared and tested by the procedure of Reference Example 1, except that human cadaver skin was employed in place of the mouse skin, and 4.0 wt % of norethisterone acetate (NETA) was employed together with the absorption promoter listed in Table 2. The results are shown in Table 2 and FIG. 4.

TABLE 2
Permeation rates of NETA across human cadaver skin when
conventional absorption promoters are used

| Reference | NETA | Absorption | |
|---|---|---|---|
| Example | (wt %) | Promoter (wt %) | SPR |
| 2-1 | 4.0 | — | 0.18 ± 0.03 |
| 2-2 | 4.0 | sorbitan monolaurate | 0.72 ± 0.09 |
| | | (15) | |
| 2-3 | 4.0 | Squalene | 0.43 ± 0.03 |
| | | (15) | |
| 2-4 | 4.0 | diethylene glycol | 0.52 ± 0.04 |
| | | monoethyl ether | |
| | | (15) | |

FIG. 4 illustrates the time-dependent changes in the cumulative amount of norethisterone acetate transported across human cadaver skin as a function of the absorption promoter used.

As can be seen from Table 2 and FIG. 4, the compositions containing sorbitan monolaurate (Reference Example 2-2), squalene (Reference Example 2-3) and diethylene glycol monoethyl ether (Reference Example 2-4) as the absorption promoter enhanced the permeation rate of NETA across human cadaver skin to an extent that is significantly lower than that determined for the hairless mouse skin. Further, drug crystal formation was observed in all but Reference Example 2-4, wherein diethylene glycol monoethyl ether was employed as the absorption promoter.

### Reference Example 3

### Preparation and Testing of Transdermal Administration Compositions Containing Conventional Absorption Promoters (Human Cadaver Skin)

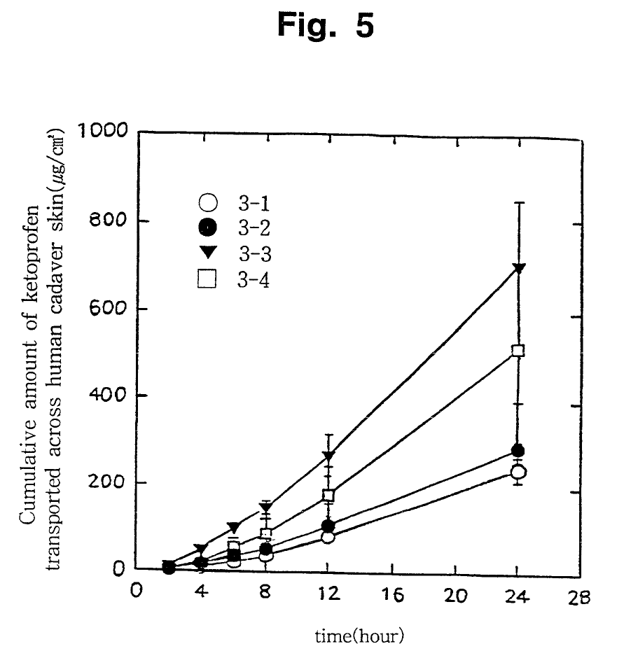

Four transdermal delivery compositions were prepared and tested by the procedure of Reference Example 1, except that human cadaver skin was employed in place of the mouse skin, and 3.0 wt % of ketoprofen (KF) was employed as a drug together with the absorption promoter listed in Table 3. The results are shown in Table 3 and FIG. 5.

TABLE 3
Permeation rates of KF across human cadaver skin when
conventional absorption promoters are used

| Reference | KF | Absorption | |
|---|---|---|---|
| Example | (wt %) | Promoter (wt %) | SPR |
| 3-1 | 3.0 | propyleneglycol(10) | 11.92 ± 1.13 |
| | | sorbitan monolaurate(10) | |
| 3-2 | 3.0 | propyleneglycol | 13.41 ± 0.40 |
| | | monolaurate(10) | |
| | | propyleneglycol(15) | |
| 3-3 | 3.0 | propyleneglycol(10) | 32.18 ± 5.71 |
| | | lauric acid(10) | |
| 3-4 | 3.0 | sorbitan monolaurate(10) | 24.17 ± 6.97 |
| | | propyleneglycol(10) | |
| | | polyvinylpyrrolidone(10) | |

FIG. 5 represents the time-dependent changes in the cumulative amounts of ketoprofen transported across human cadaver skin as a function of absorption promoter used.

As can be seen from Table 3 and FIG. 5, the compositions containing propyleneglycol and lauric acid (Reference Example 3-3), and sorbitan monolaurate, propyleneglycol and polyvinylpyrroridone (Reference Example 3-4) as the absorption promoter exhibited high permeation rates. However, crystal of the drugs and bubble were observed in the above Reference Examples 3-3 and 3-4, respectively.

### Examples 1 to 3 and Comparative Examples 1 to 6

Nine transdermal delivery compositions were prepared and tested by the procedure of Reference Example 1, except that human cadaver skin as well as 0.4 wt % of estradiol and 2.7 wt % of norethisterone acetate were employed together with the absorption promoters listed in Table 4.

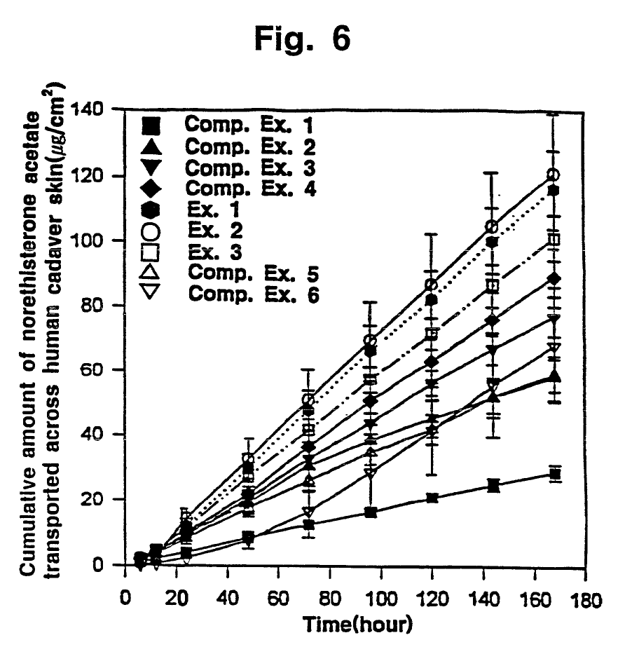

As a positive control, a commercially available reservoir type formulation, i.e., Estragest® (EG, CibaGeigy, Swiss) was used in Comparative Example 6. The results are shown in Table 4 and FIG. 6.

TABLE 4
Permeation rates of ED and NETA across human cadaver skin

| | ED | NETA | SML | TC | SPR | SPR |
|---|---|---|---|---|---|---|
| No. | (wt %) | (wt %) | (wt %) | (wt %) | (ED) | (NETA) |
| Comp. | 0.4 | 2.7 | — | — | 0.08 | 0.17 |
| Ex. 1 | | | | | ±0.02 | ±0.01 |
| Comp. | 0.4 | 2.7 | 5 | — | 0.19 | 0.41 |
| Ex. 2 | | | | | ±0.01 | ±0.05 |
| Comp. | 0.4 | 2.7 | 10 | — | 0.22 | 0.46 |
| Ex. 3 | | | | | ±0.02 | ±0.03 |
| Comp. | 0.4 | 2.7 | 15 | — | 0.27 | 0.51 |
| Ex. 4 | | | | | ±0.06 | ±0.02 |
| Ex. 1 | 0.4 | 2.7 | 10 | 10 | 0.23 | 0.72 |
| | | | | | ±0.01 | ±0.06 |
| Ex. 2 | 0.4 | 2.7 | 10 | 5 | 0.23 | 0.75 |
| | | | | | ±0.03 | ±0.06 |
| Ex. 3 | 0.4 | 2.7 | 5 | 10 | 0.26 | 0.62 |
| | | | | | ±0.03 | ±0.03 |
| Comp. | 0.4 | 2.7 | — | 10 | 0.17 | 0.35 |
| Ex. 5 | | | | | ±0.04 | ±0.02 |
| Comp. | | | | | 0.15 | 0.45 |
| Ex. 6 | | | | | ±0.04 | ±0.09 |

SML: sorbitan monolaurate,
TC: diethylene glycol monoethyl ether

FIG. 6 exhibits the time-dependent changes in the cumulative amount of norethisterone acetate transported across human cadaver skin as a function of the contents of sorbitan monolaurate and diethylene glycol monoethyl ether.

As can be seen from Table 4 and FIG. 6, the inventive compositions containing a mixture of diethylene glycol monoethyl ether (TC) and sorbitan monolaurate (SML) having a TC to SML weight ratio in the range of 0.5 to 2 (Examples 1, 2 and 3) as the absorption promoter exhibited markedly enhanced permeation rates of the drugs across human cadaver skin, as compared with those observed in Comparative Examples 1-6. The permeation rates follow apparent zero-order kinetics. Further, drug crystals were not observed in Examples 1, 2 and 3.

### Example 4 and Comparative Examples 7 to 10

Five transdermal delivery compositions were prepared and tested by the procedure of Reference Example 1, except that human cadaver skin and 3.5 wt % of testosterone were employed together with the absorption promoters listed Table 5.

TABLE 5
Permeation rates of testosterone across human cadaver skin

| | Testosterone | Absorption Promoter | |
|---|---|---|---|
| No. | (wt %) | (wt %) | SPR |
| Comp. Ex. 7 | 3.5 | — | 0.70 |
| | | | ±0.32 |
| Comp. Ex. 8 | 3.5 | SML (20) | 3.07 |
| | | | ±1.22 |
| Comp. Ex. 9 | 3.5 | Propyleneglycol | 2.21 |
| | | monolaurate (20) | ±0.04 |
| Comp. Ex. 10 | 3.5 | TC (20) | 2.03 |
| | | | ±0.25 |
| Ex. 4 | 3.5 | TC(10) and SML(10) | 3.57 |
| | | | ±0.77 |

As the results in Table 5 show, the inventive composition containing a mixture of diethylene glycol monoethyl ether (TC) and sorbitan monolaurate (SML) (Example 4) as the absorption promoter exhibited a markedly higher permeation rate than those observed when TC or SML was used alone (Comparative Examples 8 and 9).

### Examples 5 to 7 and Comparative Examples 11 to 12

Five transdermal delivery compositions were prepared and tested by the procedure of Reference Example 1, except that human cadaver skin and 0.8 wt % of estradiol were employed together with the absorption promoters listed Table 6.

TABLE 6
Permeation rates of estradiol across human cadaver skin as a
function of the absorption promoter used

| | ED | Absorption Promoter | |
|---|---|---|---|
| No. | (wt %) | (wt %) | SPR |
| Comp. Ex. 11 | 0.8 | — | 0.15 ± 0.01 |
| Comp. Ex. 12 | 0.8 | SML (10) | 0.31 ± 0.02 |
| Ex. 5 | 0.8 | TC(10) and SML(2.5) | 0.54 ± 0.06 |
| Ex. 6 | 0.8 | TC(10) and SML(5) | 0.58 ± 0.01 |
| Ex. 7 | 0.8 | TC(10) and SML(10) | 0.42 ± 0.03 |

As the results in Table 6 show, the inventive compositions containing a mixture of diethylene glycol monoethyl ether(TC) and sorbitan monolaurate (SML) having a TC to SML ratio ranging from 1 to 4 (Example Nos. 5, 6 and 7) exhibited enhanced permeation rates estradiol through human cadaver skin.

### Example 8 and Comparative Examples 13 to 14

A transdermal delivery composition was prepared and tested by the procedure of Reference Example 1, except that human cadaver skin as well as 3.0 wt % of ketoprofen(KF) was employed together with the absorption promoters listed in Table 7.

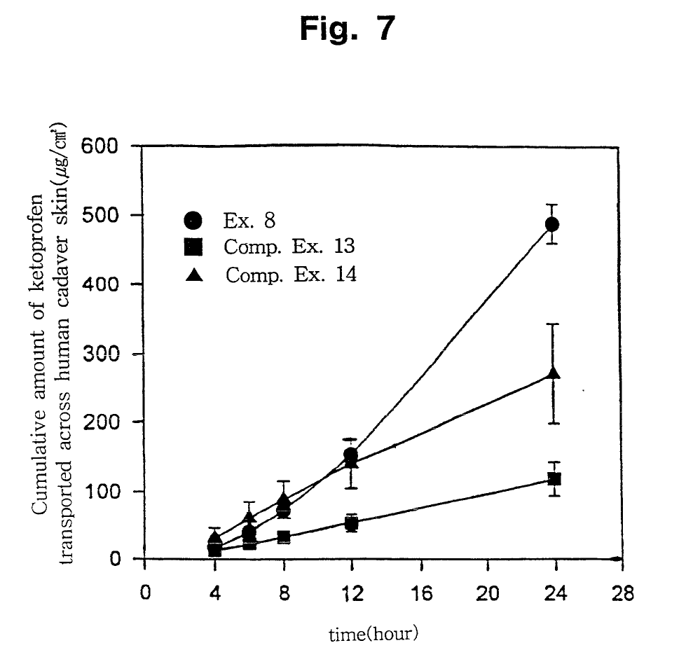

As positive controls, commercially available plasters of ketoprofen, i.e., Ketotop® (Pacific Pharmaceutical Co., LTD, Korea) and Kefentech® (Jeil Pharmaceutical Co., LTD., Korea) were used in Comparative Examples 13 and 14, respectively. The results are shown in Table 7 and FIG. 7.

TABLE 7
Permeation rates of KF across human cadaver skin

| | Amount of KF | Absorption Promoter | |
|---|---|---|---|
| No. | (wt %) | (wt %) | SPR |
| Ex. 8 | 30 mg | TC(10) and SML(10) | 22.76 |
| | (3 wt %) | | ±0.96 |
| Comp. Ex. 13 | 30 mg | | 5.24 |
| | | | ±0.83 |
| Comp. Ex. 14 | 30 mg | | 11.95 |
| | | | 2.23 |

FIG. 7 compares the time-dependent changes in the cumulative amount of ketoprofen transported across human cadaver skin in the experiments employing the inventive formulation and commercially available formulations.

As the results in Table 7 and FIG. 7 show, the inventive composition containing a mixture of diethylene glycol monoethyl ether (TC) and sorbitan monolaurate (SML) (Example 8) as the absorption promoter exhibits higher permeation rate than those of Comparative Examples 13 and 14 by a factor of 2 to 4.

### Example 9 and Comparative Example 15

Two transdermal delivery compositions were prepared and tested by the procedure of Reference Example 1, except that human cadaver skin as well as 3.0 wt % of ibuprofen was employed together with the absorption promoters listed in Table 8.

The results are shown in Table 8.

TABLE 8
Permeation rates of ibuprofen across human cadaver skin

| | Ibuprofen | Absorption Promoter | |
|---|---|---|---|
| No. | (wt %) | (wt %) | SPR |
| Comp. Ex. 15 | 3.0 | | 2.01 |
| | | | ±0.74 |
| Ex. 9 | 3.0 | TC(10) and SML(10) | 13.03 |
| | | | ±2.27 |

As the results in Table 8 show, the inventive composition containing a mixture of diethylene glycol monoethyl ether (TC) and sorbitan monolaurate (SML) (Example 9) as the absorption promoter exhibited a markedly higher permeation rate than that observed in Comparative Example 15.

### Example 10 and Comparative Example 16

Two transdermal delivery compositions were prepared and tested by the procedure of Reference Example 1, except that human cadaver skin as well as 3.0 wt % of flurbiprofen was employed together with the absorption promoters listed in Table 9.

The results are shown in Table 9.

TABLE 9
Permeation rates of Flurbiprofen across human cadaver skin

| | Flurbiprofen | Absorption Promoter | |
|---|---|---|---|
| No. | (wt %) | (wt %) | SPR |
| Comp. Ex. 16 | 3.0 | | 1.68 |
| | | | ±0.56 |
| Ex. 10 | 3.0 | TC(10) and SML(10) | 8.58 |
| | | | ±3.27 |

As the results in Table 9 show, the inventive composition containing a mixture of diethylene glycol monoethyl ether(TC) and sorbitan monolaurate (SML) (Example 10) as the absorption promoter exhibited a markedly higher permeation rate than that observed in Comparative Example 16.

### Example 11 and Comparative Example 17

Two transdermal delivery compositions were prepared and tested by the procedure of Reference Example 1, except that human cadaver skin as well as 3.0 wt % of diclofenac was employed together with the absorption promoters listed in Table 10.

The results are shown in Table 10.

TABLE 10
Permeation rates of diclofenac across human cadaver skin

| | Diclofenac | Absorption Promoter | |
|---|---|---|---|
| No. | (wt %) | (wt %) | SPR |
| Comp. Ex. 17 | 3.0 | | 0.78 |
| | | | ±0.29 |
| Ex. 11 | 3.0 | TC(10) and SML(10) | 3.77 |
| | | | ±0.75 |

As the results in Table 10 show, the inventive composition containing a mixture of diethylene glycol monoethyl ether (TC) and sorbitan monolaurate (SML) (Example 11) as the absorption promoter exhibited a markedly higher permeation rate than that observed in Comparative Example 17.

### Example 12 and Comparative Example 18

Two transdermal delivery compositions were prepared and tested by the procedure of Reference Example 1, except that human cadaver skin as well as 3.0 wt % of naproxen was employed together with the absorption promoters listed in Table 11.

The results are shown in Table 11.

TABLE 11
Permeation rates of naproxen across human cadaver skin

| | Naproxen | Absorption Promoter | |
|---|---|---|---|
| No. | (wt %) | (wt %) | SPR |
| Comp. Ex. 18 | 3.0 | | 2.34 ± 0.28 |
| Ex. 12 | 3.0 | TC(10) and SML(10) | 11.74 ± 4.02 |

As the results in Table 11 show, the inventive composition containing a mixture of diethylene glycol monoethyl ether (TC) and sorbitan monolaurate (SML) (Example 12) as the absorption promoter exhibited a markedly higher permeation rate than that observed in Comparative Example 18.

While the invention has been described with respect to the above specific embodiments, it should be recognized that various modifications and changes may be made to the invention by those skilled in the art which also fall within the scope of the invention as defined by the appended claims.

What is claimed is:

1. A composition for the transdermal administration of a drug, comprising: 0.05 to 50 wt % of the drug based on the total weight of the composition; 1 to 30 wt % of an absorption promoter consisting of a diethylene glycol ether and a sorbitan ester in a weight ratio ranging from 1:4 to 4:1, based on the total weight of the composition; and a pharmaceutically acceptable adhesive matrix.

2. The composition of claim 1, wherein the weight ratio of the diethylene glycol ether and the sorbitan ester ranges from 1:2 to 2:1.

3. The composition of claim 1, wherein the diethylene glycol ether is diethylene glycol monoethyl ether, diethylene glycol monomethyl ether or a mixture thereof.

4. The composition of claim 1, wherein the sorbitan ester is sorbitan monolaurate, sorbitan monooleate or a mixture thereof.

5. The composition of claim 1, wherein the amount of the drug ranges from 0.1 to 20 wt % based on the total weight of the composition.

6. The composition of claim 1, wherein the drug is an estrogen, progestogen, androgen, non-steroidal anti-inflammatory drug or a mixture thereof.

7. The composition of claim 6, wherein the amount of the androgen ranges from 1 to 10 wt % based on the total weight of the composition.

8. The composition of claim 6, wherein the amount of the non-steroidal anti-inflammatory drug ranges from 1 to 10 wt % based on the total weight of the composition.

9. The composition of claim 6, wherein the drug consists of 0.05 to 5 wt % of estrogen and 1 to 10 wt % of progestogen, each based on the total weight of the composition.

10. The composition of claim 6, wherein the estrogen is estradiol, ethynyl estradiol or estradiol ester.

11. The composition of claim 6, wherein the progestogen is norethisterone, norethisterone acetate, medroxyprogesterone acetate, desogestrel, gestaten or levonorgestrel.

12. The composition of claim 6, wherein the androgen is testosterone, testosterone propionate, testosterone enanthate, testosterone cypionate, methyltestosterone or dehydroepiandrosterone.

13. The composition of claim 6, wherein the non-steroidal anti-inflammatory drug is ketoprofen, flurbiprofen, methylsalicylate, salicylic acid, ibuprofen, indomethacin, diclofenac sodium, flufenamic acid, naproxen, mefenamic acid, fenoprofen, fenclofenac, piroxicam or a mixture thereof.

14. The composition of claim 1, wherein the amount of the absorption promoter ranges from 5 to 25 wt % based on the total weight of the composition.

15. The composition of claim 1, wherein the adhesive matrix is a polyacrylate adhesive, polyisobutylene or silicon rubber.

16. A transdermal formulation for the transdermal administration of a drug, comprising: a protective backing layer; a drug reservoir layer containing the composition of claim 1, which is placed on the protective backing layer, one side of which is laminated on the protective backing layer; and a removable peel layer attached to the other side of the drug reservoir layer.

17. The formulation of claim 16, which further comprises a supplementary adhesive layer.
